(19) United States
(12) Patent Application Publication
(10) Pub. No.: US 2021/0353272 A1
(43) Pub. Date: Nov. 18, 2021

(54) INTELLIGENT HANDPIECE

(71) Applicant: Aesculap AG, Tuttlingen (DE)

(72) Inventors: Roland-Alois Högerle, Tuttlingen (DE); Thomas-Erwin Kahler, Seitingen-Oberflacht (DE); Martin Machill, Rietheim-Weilheim (DE)

(21) Appl. No.: 17/283,984

(22) PCT Filed: Oct. 10, 2019
(86) PCT No.: PCT/EP2019/077492

§ 371 Date: Apr. 9, 2021

(30) Foreign Application Priority Data
Oct. 11, 2018 (DE) 10 2018 125 181.5

(51) Int. Cl. A61B 17/00; B25J 13/00; B25J 13/06; G08C 17/02

(52) U.S. Cl. CPC A61B 17/00; B25J 13/006; H02J 50/001; G08C 17/02; B25J 13/06

(57) ABSTRACT

A communication device for a surgical coupling system, which coupling system has first and second coupling devices, each having at least two electrical coupling contacts. The coupling system has a disconnected state, an OFF state, in which at least one electrical coupling contact of the first coupling device and of the second coupling device are disengaged, and an ON state, in which the at least one electrical coupling contact of the first coupling device and of the second coupling device are conductively in contact. The communication device includes at least one usage part in which an electric motor is accommodated, and a control unit for controlling the electric motor. An integrated intelligent device maintains communication between the usage part and the control unit in the OFF state and in the ON state. A supply cable has at most three lines between the usage part and the control unit.

### CROSS-REFERENCE TO RELATED APPLICATION(S)

This application is the United States national phase entry of International Application No. PCT/EP2019/077492, filed Oct. 10, 2019, and claims the benefit of priority of German Application No. 10 2018 125 181.5, filed Oct. 11, 2018. The contents of International Application No. PCT/EP2019/077492 and German Application No. 10 2018 125 181.5 are incorporated by reference herein in their entireties.

### FIELD

The present invention relates to a communication device, in particular for a surgical coupling system or respectively of a surgical coupling system.

### BACKGROUND

As is already known from DE 10 225 857 A1, a control unit can be connected to several application parts/handpieces via a surgical coupling system, which will be described in more detail later. In surgery, different motor units with the same or different motors, in particular surgical motors, are used depending on the application. Depending on the required speed, corresponding motor types are selected and integrated into an application part. Owing to the coupling system described in DE 10 225 857 A1, only one control unit is now required for at least two application parts/handpieces. Thus, in the operating area, the reduction in the number of control devices can increase clarity and reduce the probability of incorrect operation of the control units.

An electrical switching device is known from DE 10 2011 050 192 A1, which is based on the prior art described above and describes the surgical coupling system in more detail. The surgical coupling system consists of a first coupling device and a second coupling device, which are designed as a (plug-in) connection in order to connect an application part/handpiece to a control unit/control device. The first coupling device is located at one end of a supply cable, the other end of which is connected to the control unit and connects it to the application part. The second coupling device is part of the application part in which the electric motor is integrated. Here, the first coupling device and the second coupling device each comprise at least two mechanically engageable electric coupling contacts.

The integrated electric motor has three motor windings which are connected to each other in a star circuit. Each motor winding is connected to the second coupling device via a respective line. This means, with respect to an example in which each coupling device is formed with four electric coupling contacts each, that two electric coupling contacts of the second coupling device are connected to one respective line. The other two electric coupling contacts of the second coupling device are connected in parallel and connected to the third line. A resistor element, which forms a coding element for coding the type of handpiece, is interposed in front of one of the two parallel-connected, electric coupling contacts. Via this signal path, the type of electric motor integrated in the application part can be determined by means of a resistance measurement via the resistance element, and this information can be passed on to the control unit via the signal path in order to drive the motor in accordance with this information.

Here, the coupling system defines three different positions/states. The first and second coupling devices are therefore in a separated position when they are completely separated from each other. This means that none of the electric coupling contacts of the first coupling device are in contact with the electric coupling contacts of the second coupling device, nor are they mechanically engaged with each other. In an OFF position, at least one first electric coupling contact of the first coupling device is disengaged from at least one first electric coupling contact of the second coupling device. That is, when the first and second coupling devices are each formed with four electric coupling contacts, and two electric coupling contacts of the first coupling device are engaged with two electric coupling contacts of the second coupling device, the coupling system is in an OFF position. The coupling device is in an ON position when all electric coupling contacts of the first coupling device are in electrically conductive contact with all corresponding electric coupling contacts of the second coupling device.

Thus, the coupling system can be used as a switching device and has a dual function. On the one hand, it can establish a mechanical connection between the handpiece and, for example, the supply line. On the other hand, it can also be used as a switching device in order, for example, to be able to supply motor windings of an electric motor of the handpiece with power as required.

Furthermore, this allows the additional requirement in which the type of handpiece can be automatically retrieved by a control and/or regulation device of a drive system. However, this communication can only take place in the OFF position described above and the signal path has to be routed to the control unit via various stranded wires/lines, or the motor winding and several interfaces, such as plug contacts. The reason for this is that in the ON position, when both of the parallel-connected, electric coupling contacts are in contact, the signal path is short-circuited via the resistor element and thus information can no longer be exchanged between the application part and the control unit. The path of the signal path is also disadvantageous, since it can lead to distortions, faulty transmissions and time delays, and thus in total to tolerances, which can then be interpreted as a false signal at the control unit. In order to maintain such a communication path in the ON position and thus to enable an exchange of information between the application part and the control unit, a solution can be found by increasing the number of supply lines/stranded wires for the electric motor.

### SUMMARY

Therefore, the object of the invention is to provide a communication device for a surgical coupling system according to the preceding description, which enables continuous, reliable and safe communication between the application part and the control unit without the use of further lines and realizes this in a simple system.

The invention is based on the general idea of providing continuous/prolonged communication/connection for a surgical coupling system connecting at least one application part/handpiece and a control unit. The application part houses an electric motor for driving a surgical tool attached/inserted to the application part. The application part can also be designed, for example, as an interface for connecting to a robot.

According to the underlying general idea, there is a communication connection between the application part and the control unit, which provides for an exchange of information both in the OFF state and in the ON state, in order to read out from the data collected in the control unit all the information of all the individual application parts used in the control unit. This information can be an evaluation of the basic information, such as the number of uses, for example in the operating room, the total run time, the run time per use as well as the run time in clockwise/anticlockwise operation, start/stop cycles, current consumption, etc. of the respective application part. In addition, the evaluation of the sensor signals carried out in the control unit covers the frequency of the tool type used, humidity conditions, temperatures, number of reprocessings, etc. and even statistics on all application parts used.

For maintaining the communication connection between the application part and the control unit for transmitting various information and sensor signals of the application part, no further stranded wires/lines are provided in the supply cable/connection cable. The number of lines is determined by the number of motor windings, preferably three motor windings, of the electric motor.

The use of a communication device integrated in the application part provides a reliable and more secure information transmission, which can be used in both an OFF and an ON state, as defined in the prior art above. This communication connection/signal path can thus be maintained for a longer period of time or even continuously.

In detail, the communication device for a surgical (electric) coupling system has an intelligent device integrated in the application part, which is adapted to maintain communication between the application part and the control unit in the OFF state and/or in the ON state. A supply cable between the application part and the control unit has a maximum of three wires, corresponding to the number of motor windings of the electric motor. An intelligent device is generally understood to mean a device that deals with the automation of intelligent behavior and machine learning. The ‘intelligence’/intelligent device of the present invention has various basic information of the application part, such as serial number, date of manufacture, maintenance data, etc. The intelligent device passes this basic information to the control unit. In the control unit, the information is collected/stored and evaluated/processed. The design of the communication device allows the number of required electrical contact elements to be minimized, in particular to the number of existing motor windings of the electric motor. Further lines, for example in order to actuate the electric motor in conventional drive systems and, if necessary, to request information on its type or design, can be dispensed with in the present invention. For controlling and/or regulating the electric motor, it is further advantageous if the control unit is configured in particular to cooperate with the surgical handpiece/application part in such a way that it can automatically request and recognize the type of application part.

It is preferred when the intelligent device is connected directly into a signal path between one of the parallel-connected, electric coupling contacts of the second coupling device and a corresponding motor winding of the electric motor. Thus, the intelligent device can be continuously powered and can continuously communicate with the control unit in the OFF position.

It is further advantageous if the communication between the application part and the control unit is wireless. In this way, communication in the ON position is possible despite a short-circuited signal path, according to the preceding description from the prior art. Such wireless transmission methods are data transmission methods that use free space as a transmission medium and dispense with a cable in the form of an electrical conductor or an optical waveguide. Preferably, transmission methods such as those provided under the federally registered trademark BLUETOOTH® or WLAN are used. Alternatively, transmission methods such as those provided under the federally registered trademarks ZIGBREE®, NFC® WIBREE™ or others can be used, depending on the amount of data and the required range.

Preferably, an energy storage device is provided in or attached to the application part that is electrically connected to the intelligent device. In the OFF state, the additional energy storage device can be charged via the supply line and the intelligent device can communicate wirelessly with the control unit either continuously or for a longer period in the ON state. Even an application part that has been completely removed, or is in the separated state, is able to continue communicating with the control unit until the energy storage device is exhausted. At least a rechargeable battery can be used as an energy storage device, e.g. lithium-ion or lithium-polymer batteries. The use of small and lightweight energy storage devices is advantageous here.

It is advantageous if the intelligent device has a discharge protection fuse. Deep discharge of an energy storage device can cause different types of damage depending on the type of energy storage device. Thus, it is advantageous if the discharge protection fuse ensures that the voltage does not drop below a discharge cutoff voltage. The discharge protection fuse provided in the intelligent device therefore has the object to prevent this in the ON state and separated state. Here, the discharge protection fuse ensures electrical disconnection of the energy storage device from the intelligent device when, depending on the type of energy storage device, a fixed voltage is reached up to which the energy storage device is allowed to discharge.

Preferably, the intelligent device has several inputs for different sensor signals. Here, sensors are provided, in particular temperature sensors, sensors for whether a tool is present or respectively which type of tool is present, humidity sensor, sensors for recognition of reprocessing, etc. from the application part. These sensors thus provide/send further information to the control unit by means of sensor signals over a period of time until the energy storage device is exhausted or has reached the set voltage, as described above.

According to a further aspect of the invention, the intelligent device is configured to communicate with the control unit and/or to communicate among several application parts. In other words, this means that the intelligent device is capable of constantly communicating and exchanging information with the control unit and also among the other application parts. For example, by means of this communication among the application parts themselves, a prescribed order in their use during an operation could be ensured. Thus, error-prone sequences of operations can be supported and process reliability can be increased. Such a communication between the application parts can also serve to prevent errors, to increase patient safety and to reduce the surgeon's workload. In addition, it is advantageous if such communication takes place in real time.

Alternatively, the voltage supply to the intelligent device can be indirect, in particular inductive. Due to the proximity of the intelligent device to the energy storage device given in the application part, inductive coupling offers an alternative to direct voltage supply. Inductive coupling is understood to mean the mutual magnetic influence of two or more spatially adjacent electrical circuits by electromagnetic induction as a result of a change in the magnetic flux. Here, a current-carrying (first) conductor loop causes the generation of a magnetic flux density in its spatial environment. Inductive energy transmission has a comparatively high efficiency in the close range. In this respect, it is advantageous if the distance between a transmitter, in this case the energy storage device, and a receiver, in this case the intelligent device, is kept as short as possible. Alternatively, another option would be to charge the energy storage device wirelessly by means of inductive coupling.

Advantageously, the intelligent device is in the center of a coil, which is positioned in the signal path and is necessary for the implementation of inductive coupling in order to be able to guarantee an optimal inductive voltage supply.

According to another aspect of the invention, the intelligent device is powered by harvesting from the lines of the power supply of the electric motor or scattering magnetic fields of the electric motor. Harvesting means that energy is obtained from the environment. Possible sources are e.g. temperature differences, movement (e.g. pressing a switch, moving machine parts), light (e.g. ambient light), which contribute to energy generation with a corresponding energy converter with different power parameters.

### BRIEF DESCRIPTION OF THE DRAWING FIGURES

FIG. 1 is a schematic circuit diagram illustrating the coupling system according to a separated state of the present disclosure;

FIG. 2 is a schematic circuit diagram illustrating the coupling system according to an OFF state of the present disclosure;

FIG. 3 is a schematic circuit diagram illustrating the coupling system according to an ON state of the present disclosure;

FIG. 4 is a schematic representation in sections of the application part according to a first configuration example;

FIG. 5 is a schematic representation in sections of the application part according to a second configuration example;

FIG. 6 is a schematic representation in sections of the application part according to a third configuration example; and

FIG. 7 is a schematic representation in sections of several application parts.

### DETAILED DESCRIPTION

Hereinafter, configuration examples of the present disclosure are described on the basis of the associated figures. Identical or functionally equivalent features are provided with the same reference signs in the individual figures and are expediently not described more than once.

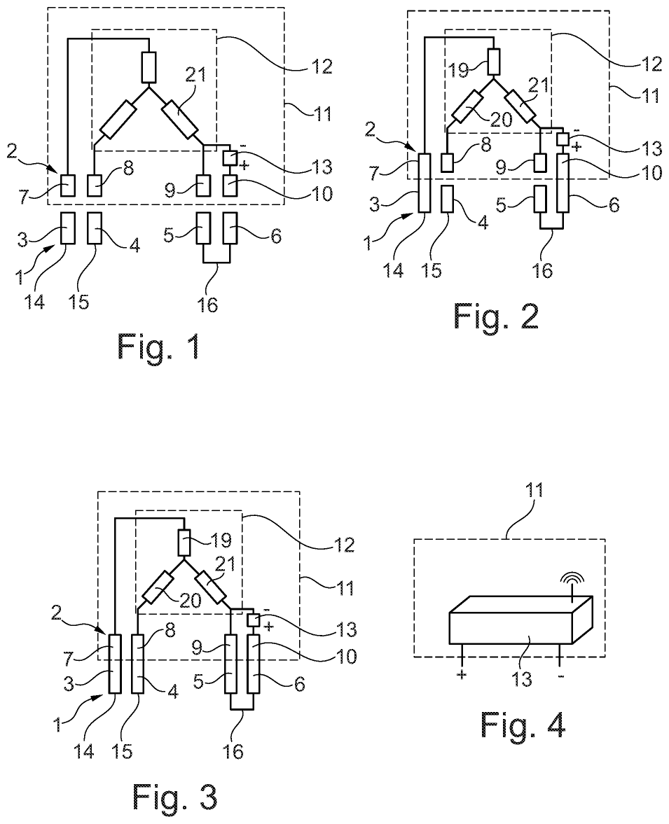

FIG. 1 is a schematic circuit diagram illustrating a coupling system according to a separated state. The first or male, surgical coupling device 1 and the second or female, surgical coupling device 2 are completely disconnected from each other, i.e., disengaged. Each of the two coupling devices 1 and 2 has respectively four electric coupling contacts. The first coupling device 1 has electric coupling contacts 3, 4, 5 and 6, and the second coupling device 2 has electric coupling contacts 7, 8, 9 and 10.

An application part/handpiece 11 is shown as a schematic circuit diagram. An electric motor 12 and an intelligent device 13 are housed/integrated in the application part 11. Furthermore, the application parts 11 may differ not only externally, but also in their internal structure. This means, for example, that the electric motors 12 installed in the application parts 11 can be of different types and differ, for example, in their characteristics, such as minimum speed, maximum speed, maximum current and maximum torque.

Furthermore, the application part 11 is equipped with the electric coupling contacts 7, 8, 9 and 10 of the second coupling device 2. The electric motor 12 serves as a drive system for a surgical tool attachable and detachably connected to the application part 11. A maximum of three lines 14, 15 and 16 form a supply cable. The three lines 14, 15 and 16 supply power to the motor windings 19, 20 and 21 of the electric motor 12.

The coupling contact 7 is connected to the motor winding 19 in an electrically conductive manner. The motor winding 19 is connected in a star shape with the motor windings 20 and 21. The motor winding 20 is also connected in an electrically conductive manner to the electric coupling contact 8. The motor winding 21 is connected in an electrically conductive manner on the one hand to the coupling contact 9 and on the other hand to the intelligent device 13, which is connected in series with the electric coupling contact 10. The electric coupling contact 9 is connected in parallel with the intelligent device 13 and the electric coupling contact 10. A kind or type of the application part 11 can be unambiguously identified via the intelligent device 13. By a corresponding design or programming of the control unit, this determination/identification can be carried out automatically.

FIG. 2 is a schematic circuit diagram illustrating the coupling system according to an OFF state. In the OFF state of the coupling system, the electric coupling contacts 7 and 10 of the second coupling device are connected to the electric coupling contacts 3 and 6 of the first coupling device in an electrically conductive manner. This closes a circuit in which the coupling contact 7 is connected in series with the motor winding 19, the motor winding 20, the intelligent device 13, and the coupling contact 10. In the OFF state, the intelligent device 13 can be supplied with power by applying a voltage to the lines 14 and 16, and an exchange of information between the intelligent device 13 and the control unit is permanently possible in this state.

FIG. 3 is a schematic circuit diagram illustrating the coupling system according to an ON state. In the ON state, all electric coupling contacts 3, 4, 5 and 6 of the first coupling device 1 are engaged with the corresponding electric coupling contacts 7, 8, 9 and 10. In the ON state, the motor windings 19, 20 and 21 of the electric motor 12 can be supplied by means of the control unit in a manner adapted to the application part.

The control unit is capable of driving the electric motor 12 in a desired manner, for example, to cause a surgical tool that is detachably connected to the application part 11 to be rotated by the electric motor 12.

Hereinafter, the coupling system described above forms the basis of the following alternative configuration examples.

### First Configuration Example

FIG. 4 is a schematic representation in sections of the application part 11 according to a first configuration example. The intelligent device 13 integrated in the application part 11, which is directly connected in series in the signal path between the motor winding 21 and the electric coupling contact 10, is capable of communicating both in the OFF state according to FIG. 2 continuously and in the ON state according to FIG. 3 and/or in the separated state according to FIG. 1 for a longer period of time.

FIG. 4 shows the intelligent device 13 of the application part 11 with the possibility of using a wireless/cableless information transmission method, such as Bluetooth, WLAN, etc. Via this connection, the intelligent device 13 sends information to the control unit. The control unit collects the received information, processes and evaluates it in order to control/regulate the electric motor 12 according to the information.

### Second Configuration Example

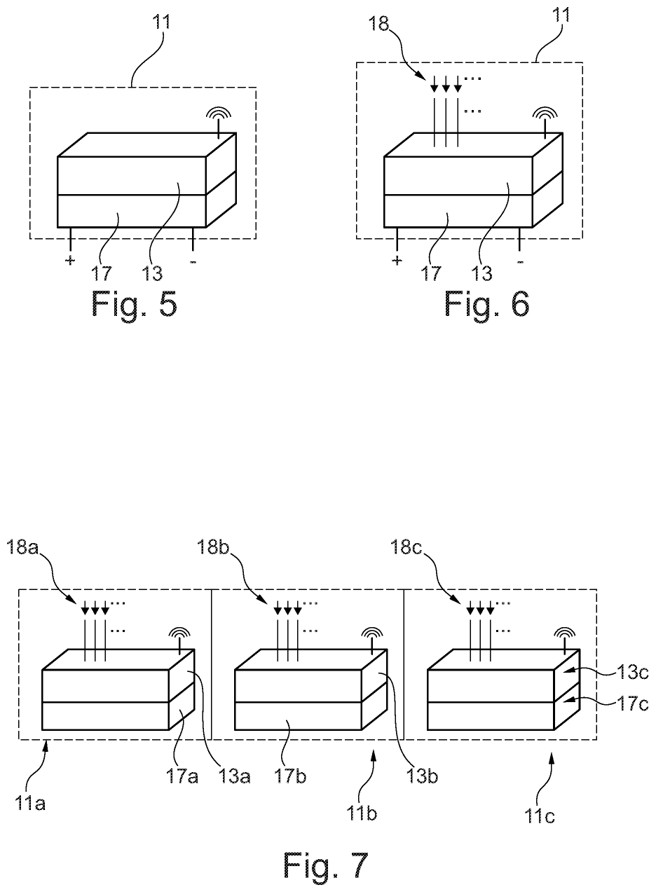

FIG. 5 shows schematic representation in sections of the application part 11 according to a second configuration example. The intelligent device 13 is connected to and/or attached to an energy storage device 17. The energy storage device 17 supplies power to the intelligent device 13, so that continuous or, in the ON state, wireless communication with the control unit is possible for a longer period of time. Also, a further sustained communication connection between a completely removed application part 11 and the control unit is possible until the energy storage device 17 is exhausted.

The energy storage device 17 can be charged in the OFF state to then supply energy to the intelligent device 13 in the ON state and/or in the separated position. According to the introductory part, this energy transfer may be direct or indirect.

When at least one energy storage device 17 is used, the intelligent device 13 has a discharge protection fuse (not shown) which prevents deep discharge and thus damage to the energy storage device 17 in the ON state.

### Third Configuration Example

FIG. 6 is a schematic representation in sections of the application part 11 according to a third configuration example. According to the second configuration example, the intelligent device 13 is connected to or attached to an energy storage device 17. The application part 11 is preferably equipped with sensors. Furthermore, the intelligent device 13 has several signal inputs 18 for various additional sensor signals which are sent from the intelligent device 13 to the control unit. The control unit processes and evaluates the received sensor signals in order to control/drive the electric motor 12 according to the signals.

FIG. 7 is a schematic circuit diagram in sections of several application parts. FIG. 7 shows application parts 11a, 11b, and 11c, each of which houses an intelligent device 13a, 13b, and 13c. Each intelligent device 13a, 13b, and 13c is connected to or attached to an energy storage device 17a, 17b, and 17c. The intelligent device 13a has several signal inputs 18a, the intelligent device 13b has several signal inputs 18b, and the intelligent device 13c has several signal inputs 18c. The respective signal inputs 18a, 18b, and 18c provide signals from the respective sensors of the respective application parts 11a, 11b, and 11c to the respective intelligent device 13a, 13b, or 13c. It goes without saying that the number of application parts 11 is not limited to three and can be reduced or expanded as needed.

It is provided that the intelligent devices 13a, 13b and 13c each communicate with the control unit alternately and/or simultaneously in the OFF state continuously and in the ON state or separated state for a longer period of time until the respective energy storage devices 17a, 17b and 17c are exhausted. Furthermore, it is provided that the application parts 11a, 11b and 11c can also communicate with each other in order to ensure, for example, a predetermined sequence when several handpieces are used in succession. In order to be able to ensure this, communication in real time is provided.

It is understood that the configuration examples described and the drawings, which are not true to scale, are merely exemplary in character, and that in this respect modifications can be made by a person skilled in the art without departing from the scope of the description. Likewise, external shapes, dimensions and the like are not subject to any particular limitations as long as they provide and achieve the effect and functionality according to the invention.

1. A communication device for a surgical coupling system having a first surgical coupling device and a second surgical coupling device each having at least two mechanically engageable electric coupling contacts, and having a separated state, an OFF state in which at least one electric coupling contact of the first coupling device and of the second coupling device are disengaged, and having an ON state in which the at least one electric coupling contact of the first coupling device and of the second coupling device are in electrically conductive contact, the communication device further comprising:
a manually grippable application part in which an electric motor for driving a surgical tool attached to the application part is housed;
a control unit for regulating and/or controlling the electric motor;
an intelligent device incorporated in the application part, that is configured to maintain a communication between the application part and the control unit in the OFF state and in the ON state; and
a supply cable having a maximum of three lines between the application part and the control unit.

2. The communication device according to claim 1, wherein the intelligent device is directly connected in a signal path between a second electric coupling contact of the second coupling device and the electric motor.

3. The communication device according to claim 1, wherein the communication between the application part and the control unit is wireless.

4. The communication device according to claim 1, wherein an energy storage device is provided in or attached to the application part and is electrically connected to the intelligent device.

5. The communication device according to claim 4, wherein the intelligent device has a discharge protection fuse.

6. The communication device according to claim 1, wherein the intelligent device has several inputs for sensor signals.

7. The communication device according to claim 1, wherein the intelligent device is configured to communicate with the control unit and/or to communicate among several application parts.

8. The communication device according to claim 4, further comprising a voltage supply that is indirect.

9. The communication device according to claim 2, wherein the intelligent device is positioned in a center of a coil in the signal path.

10. The communication device according to claim 8, wherein the intelligent device is powered by harvesting from the lines of the voltage supply or scattered magnetic fields of the electric motor.
